(19) United States
(12) Patent Application Publication
(10) Pub. No.: US 2010/0331636 A1
(43) Pub. Date: Dec. 30, 2010

(54) METHOD FOR THE CONTINUOUS NON-INVASIVE DETERMINATION OF THE CONCENTRATION OF BLOOD CONSTITUENTS

(75) Inventors: Thomas Hübner, Jena (DE); Michael Alt, Kirchheim (DE)

Correspondence Address: MAIER & MAIER, PLLC, 1000 DUKE STREET, ALEXANDRIA, VA, 22314, US

(73) Assignee: Enverdis GmbH, Jena (DE)

(21) Appl. No.: 12/532,726

(22) PCT Filed: Mar. 20, 2008
(86) PCT No.: PCT/EP2008/053397

§ 371 Date: Nov. 20, 2009

(30) Foreign Application Priority Data
Mar. 23, 2007 (DE) 10 2007 014583.9
Mar. 23, 2007 (EP) 07104761.7

(51) Int. Cl. A61B 5/1455

(52) U.S. Cl. 600/310

(57) ABSTRACT

The invention relates to a method for the non-invasive determination of the concentration of blood constituents, in which a radiation source (12) emits several radiation beams (14), each with a different wavelength. A first photo detector (18) receives the measurement radiation (14) of each wavelength that is reflected by a body part (16) to be examined. A second photo detector (22) receives the measurement radiation (24) of each wavelength that is transmitted by the body part (16) to be examined. The measurement radiation (14) of each wavelength that is absorbed by the body part (16) to be examined is then determined on the basis of the measurement of the reflected radiation (20) by the first radiation receiver (18) and the measurement of the transmitted radiation (24) by the second radiation receiver (22). The concentration of the different constituents is calculated from the absorption of the measurement radiation (14) that has been determined for each wavelength.

The invention relates to a method for the continuous non-invasive determination of the concentration of blood constituents.

Human blood, for instance, consists by up to 38.5% of hemoglobin in the dry mass (i.e. all of the uncombined water is withdrawn) and ca. 15% in the moist matter (i.e. in the physiological normal condition). Hemoglobin in turn comprises the following constituents:
oxygen-unsaturated hemoglobin (RHb)
oxygen-saturated hemoglobin (O₂Hb)
carboxy hemoglobin (COHb)
methemoglobin (MetHb).

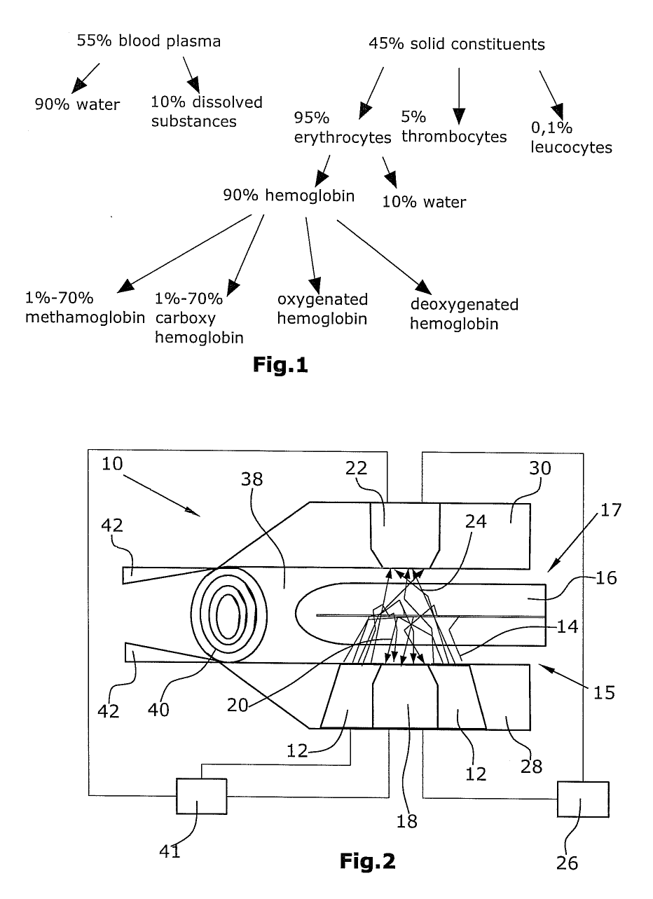

The composition of the human blood is exemplified in FIG. 1. For medical purposes, it is often required to determine the concentration of the hemoglobin and particularly the concentration of the above mentioned four hemoglobin derivates. However, the invention can also be used for determining the concentration of further constituents of the blood. Thus, all of the features mentioned in connection with hemoglobin can also be used for measurement of the concentration of other blood constituents.

For determining the hemoglobin content in the blood, it is known to use an Hb photometer. The Hb photometer comprises a capillary gap filled with a chemical reagent. A small quantity of blood, e.g. a drop of blood, will be supplied to said capillary gap and will change the light-permeability of the gap due to the chemical degrading which has occurred. The change of the light-permeability can be photometrically detected.

An apparatus of the above type has the disadvantage that, for checking the Hb value, blood has to be sampled from the patient.

Further, from US 2005/267346 A1, it is known to determine the concentration of blood constituents on the basis of absorption measurements in different wavelength ranges. However, said publication relates to an arrangement wherein electromagnetic radiation within n≧2 bands is used for determining the concentration of blood constituents. In this arrangement, each substance to be detected has to be assigned to one of said bands. In practice, however, this causes a massive restriction because, when a large number of blood constituents is to be detected, it will hardly be possible to find a corresponding number of different bands with suitable absorption structures.

U.S. Pat. No. 6,104,938 describes a method for detecting the concentration of blood constituents on the basis of an evaluation of the intensities of the transmitted and respectively reflected light of a body part on the basis of different wavelengths. The computation method is applicable only for transmitted or reflected fractions of the light but not for combined evaluation of the fractions.

It is an object of the invention to provide a method for the non-invasive determination of the concentration of a number of blood constituents, particularly of a relatively large number of blood constituents.

According to the invention, the above object is achieved by the features of claim 1.

The method for determination of the concentration of blood constituents comprises the following steps:
a. radiating a plurality of measurement radiations with respectively different wavelengths by means of a radiation source,
b. receiving the measurement radiation of a plurality of said wavelengths, as reflected by a body part to be examined, by means of a first light receiver,
c. receiving the measurement radiation of a plurality of said wavelengths, as transmitted through the body part to be examined, by means of a second light receiver,
d. determining the absorption of the measurement radiation of each wavelength, as caused by the body part to be examined, on the basis of the measurement of said reflected radiation by means of the first radiation receiver and the measurement of said transmitted radiation by means of the second radiation receiver,
e. determining the concentration of blood constituents on the basis of said determined absorption of the measurement radiation of each wavelength.

The starting point for the method of the invention is an apparatus which is suited for radiating a plurality of measurement radiations with respectively different wavelengths. Said apparatus further comprises a first light receiver for receiving the measurement radiation reflected by a body part to be examined. Further, a second light receiver is provided for receiving the measurement radiation transmitted through a body part to be examined. The body part to be examined can be e.g. a human finger. As measurement radiation, use can be made e.g. of electromagnetic radiation, such as e.g. light in the visible range and/or infrared radiation in the near-infrared range. Particularly preferred is the use of an apparatus as described in the patent application “Apparatus for detection of concentrations of blood constituents” filed by the applicant.

The invention is based on the idea that the various constituents of the blood, e.g. hemoglobin and water, will absorb radiation to different extents. Particularly, there exist specific wavelengths of the radiation, e.g. of light, in which the degree of absorption of the various blood constituents will differ very significantly. By selecting the radiation source to the effect that it will emit such a wavelength, the results obtained by the apparatus of the invention can be improved.

The theoretical foundation of the invention resides in the Lambert-Beer law

I/I₀=10^{-ECd} (1)
I exiting/transmitted intensity
I₀ incident intensity
E absorption coefficient (molar extinction) of a blood constituent (for a specific wavelength)
C concentration
d layer thickness

The Lambert-Beer law describes how the radiation intensity, when passing through an absorbing substance, will behave in dependence on the concentration of the substance. In this regard, the extinction will result from the ratio between the transmitted light and the incident light.

Thus, according to the invention, the first radiation receiver is operative to measure radiation which is reflected by the body part to be examined. Further, the second radiation receiver is operative to measure radiation which is transmitted through the body part to be examined.

Since, as initially mentioned, specific wavelengths of a radiation will be absorbed to different extents, it is rendered possible, by suitable selection of the wavelength to be used, to determine the concentration of various blood constituents on the basis of the measured degree of absorption with the aid of further calculations. As reference points in the process, use can also be made of the wavelengths of so-called isosbestic points where the blood constituents have the same degree of absorption.

Thus, the use of a radiation source for emitting different wavelengths allows for a particularly accurate measurement of the concentration of different blood constituents because a measurement can be performed exactly at those wavelengths where especially distinct differences of absorption will occur. For instance, for measuring the hemoglobin derivates, the following wavelengths have been found to be particularly advantageous:
540 nm±5 nm, 562 nm±5 nm, 573±5 nm for distinguishing carboxyhemoglobin from oxygenated hemoglobin
623±5 nm for distinguishing methemoglobin from oxygenated hemoglobin
660 nm±10 nm for distinguishing all hemoglobin derivates from each other, since, at this wavelength, all constituents have different degrees of absorption
805 nm±10 nm for distinguishing total hemoglobin from water, since, at this wavelength, only hemoglobin will be absorbed, but not water
950 nm±10 nm as a reference point because, thereat, all constituents have the same degree of absorption (isosbestic point)
1200 nm±50 nm for distinguishing water from total hemoglobin since, at this wavelength, only water will be absorbed, but not hemoglobin.

To make it possible to determine different parameters of the blood composition with respect to hemoglobin, a plurality of discrete wavelengths have to be radiated in a quasi-parallel manner into the tissue. The selection of the wavelengths, as already partially described, will be performed on the basis of the following dependencies:
1. absorption characteristics of the hemoglobin derivates
2. absorption characteristics of the water
3. penetration depth and respectively permeability of the skin for specific wavelengths
4. isosbestic point(s) in the absorption spectrum of the different derivates
5. technical possibilities of the radiation sources.

With particular preference, when selecting the wavelengths to be used, consideration is given to the optical window of the skin. In the human skin, the optical window is in a wavelength range of ca. 350 nm to 1650 nm.

Particularly, the method preferably comprises the storing of the determined absorption values for each wavelength and the repeating of the method steps mentioned up to now, wherein the determined absorption values for each wavelength will be stored for each repetition cycle. Thereafter, the individual absorption values of the emitted radiation for each wavelength will be combined so as to generate a representation of the temporal development of the absorption for each wavelength. This representation can be rendered e.g. in the form of a curve or in the form of a table.

The storing of the detected absorption values of each wavelength for each repetition cycle, the combining of the absorption values for each wavelength for representing a temporal development of the absorption and the representing of this development are performed in a computation unit in such a manner that the computation unit is not in any interdependency with the body part to be examined or with the body of a patient.

Preferably, there are used at least as many wavelengths as the number of the blood constituents to be determined. If, for instance, it is desired to determine the concentration of the four initially mentioned hemoglobin derivates as well as the water in the blood, it is required to use at least five different wavelengths. To allow for a more-precise measurement, it may be suitable, in case of further wavelengths, i.e. a total of eight wavelengths, to measure the intensity. Possible methods for determining the concentration ratios of the blood constituents are e.g. the determination by linear equation systems, the determination with the aid of a heuristic algorithm and a determination on the basis of correlation. These methods will be explained in greater detail in connection with the embodiments of the present application. In all methods, use is made of different characteristic wavelengths so that, subsequently, a volume pulse development for a plurality of individual blood constituents can be determined. In practice, for determining the concentration of individual blood constituents, it is not the entire volume pulse development that will be determined for each blood constituent but only various absorption values at decisive points of the absorption developments of the wavelengths used. These decisive points can be e.g. maxima of the absorption developments.

It is particularly preferred to perform not only a snapshot, i.e. a sole measurement, wherein absorption values at different wavelengths are determined, but a plurality of measurements at different points of time in the volume pulse developments. Particularly for detecting the pulsatile portion of the absorption development, there is required e.g. also the lowermost point of a development curve (systole) so as to then be able to determine the difference from the maximum of the development curve (diastole) because, otherwise, the DC component cannot be considered. Further, it is possible to determine a complete curve for the pulse volume development by a very large number of measurements, thus allowing also other information to be derived from this curve. For instance, instead of using the maxima, one can use the area integrals in their relationship to each other as input data for the calculation algorithm.

In most of the used wavelengths, a mixed signal (sum volume pulse) will be obtained from the absorption of all blood constituents. On the basis of the detected absorption amplitude ratios relative to each other, it is possible, with the aid of the discrete substance- and wavelength-specific extinction coefficients, to determine the concentration ratios of the blood constituents relative to each other, e.g. by use of the above calculation methods. Said extinction coefficients can be related both to the substance quantity per volume and to the mass per volume. Consequently, the ratios to be determined correspond either to the mass ratios or to the volume ratios of the individual blood constituents per volume.

Further details of an apparatus for determining the concentration of blood constituents will be described hereunder.

Said apparatus preferably comprises a computation device, connected to the first and the second radiation receiver, for computing the absorption of the emitted radiation as caused by the body part to be examined, said computation being performed on the basis of the measured reflected and transmitted fraction of the radiation. Said computation device can be e.g. a computer. Preferably, the computation of the absorption of the emitted radiation by the body part to be examined can be carried out in the computation device in such a manner than an interdependency between the computation device and the body part to be examined will not be required. For instance, the computation device can be designed as a device wherein a specific software program is run.

It is particularly preferred that the radiation source and the first radiation receiver for receiving the radiation reflected by the body part to be examined are arranged on the same side of the body part to be examined. The second radiation receiver for receiving the transmitted radiation can then be arranged opposite to the first radiation receiver on a second side of the body part to be examined.

To allow for a more-precise measurement of the hemoglobin concentration in the blood and/or to allow for a measurement of the concentration of the hemoglobin derivates, it is further preferred that the radiation source comprises a plurality of individual radiation sources of different wavelengths. Said individual radiation sources can be formed e.g. as LEDs, laser diodes or white-light LEDs with filter. This feature is of particular advantage because, at specific wavelengths, different hemoglobin derivates will have specifically outstanding differences with respect to the degree of absorption of the emitted radiation. With particular advantage, measurement is performed by use of those wavelengths where the difference in the degree of absorption for different hemoglobin derivates and respectively for further blood constituents such as e.g. water, is particularly large.

Further preferred features of the apparatus of the invention will be described hereunder.

With particular preference, the apparatus is configured to the effect that the first and the second radiation receiver are arranged opposite to each other, thus forming an accommodation chamber between the first and second radiation receiver for accommodation of the body part to be examined. In this arrangement, the radiation source and the first radiation receiver can be located in one plane.

It is particularly preferred to design the radiation source as a light source, e.g. in the form of LEDs, and to design the first and second radiation receivers as light receivers, e.g. in the form of photodiodes.

The LEDs can be positioned, preferably in a circular configuration around the first light receiver, on the first side of said accommodation chamber.

A particularly uniform illumination of the body part to be examined can be achieved in that the radiation source comprises respectively at least two individual light sources of the same wavelength, which are arranged diametrically. In case that, due to a large number of used wavelengths, it is for constructional reasons not possible to arrange two individual light sources of the same wavelength diametrically to each other, the possibility exists to use respectively one individual light source per wavelength. In this context, it is particularly preferred that the side of the individual light sources facing away from the first light receiver is lifted preferably by an angle of 15° so that the emitted radiation of the individual light sources will converge at a point where the body part to be examined, e.g. a human finger, is arranged.

To avoid detection differences, the first light receiver and the second light receiver are preferably of the same type and can be formed e.g. as photodetectors. For covering the relatively wide wavelength range of e.g. 400 nm to 1650 nm, preferred use is made of a two-color detector. This detector comprises e.g. a silicon receiver surface having a wavelength range of 400 nm to 1100 nm, such as e.g. an indium gallium arsenide receiver surface having a wavelength range of 1000 nm to 1700 nm. However, it is also possible to use a detector with e.g. three receiver surfaces of different materials. What is important is that the total range of 350 nm to 1650 nm can be detected.

For preventing the incidence of direct stray light (shunt light) on the first light receiver, the light source is separated from the first light receiver by a separating means, preferably by a light-impermeable inner and outer shell. The inner wall of the outer shell is provided with a white coating for homogenizing the light which is to be emitted. The shell can have a conical shape so that the radiation can be radiated onto a defined surface (substantially corresponding to the surface of a finger pad). Further, it can be provided that particularly the reflection sensor comprises two receivers closely adjacent to each other, so as to consider disturbing factors which can be derived from the difference between the two received signals (tissue, scattering etc.).

For reasons of hygiene and for better handling, said shells can be tightly bonded to the light source and the first light receiver. Further, the cavities located between the shells can be filled with a preferably transparent, scratch-resistant, hard and/or biocompatible adhesive. Said adhesive can terminate together with the shell in a slightly inward-curved (concave) configuration. Opposite to this arrangement, the transmissive radiation receiver is located.

The apparatus can comprise a first and a second accommodation element delimiting the accommodation chamber on its first and second side, respectively. The first and second accommodation elements can be connected to each other by a clamping mechanics in a manner allowing the apparatus to be fastened to the body part to be examined, e.g. to a finger.

With particular preference, the first and second radiation receivers are supported floatingly, thus guaranteeing an optimum contact with the body part to be examined and allowing for a consistent reproducible contact pressure. Particularly, care has to be taken that the first and second radiation receivers are in direct abutment on the to-be-examined body part and respectively on the coupling medium.

It is particularly preferred to use a glass-fiber cable for optical coupling between the LEDs and the skin and respectively between the skin and the receiver surfaces, so that the light will be focused on a smallest possible measuring area.

For improving the signal quality, the apparatus of the invention can be configured to achieve an automatic continuous tracking of the radiation intensity of the radiation sources, e.g. of the transmission LEDs, for each cable used. In case of a too small or too large output signal, the transmission power will be automatically amplified and respectively reduced. This factor has to be quantitatively reproducible so as to allow it to be included into the evaluation of the signal. The same principle can also be applied to the intensity of the radiation receivers, particularly the two-color detectors. Thus, there can be performed e.g. eight tracking processes for the eight LEDs used (eight wavelengths), and four tracking processes for the detectors used (transmissive and reflexive, respectively for two receiver surfaces).

The apparatus of the invention can be used for a large number of applications. For instance, it is possible to use the described apparatus for continuous non-invasive determination of the hemoglobin concentration. Further possible is a use of the apparatus for detection of microvascular diseases. A further possible use relates to the continuous non-invasive detection of blood pressure. The apparatus of the invention can also be used for further methods, particularly also for diagnostic or medical methods.

Particularly, the apparatus of the invention is suited for determining a volume pulse development of one or a plurality of blood constituents. From the determined volume pulse developments and particularly from the shape of a volume pulse development, further medical results, such as e.g. the blood pressure of a patient or information on the presence of microvascular diseases, can be derived. The volume pulse development of an individual blood constituent can be detected e.g. by use of a sole wavelength. In this situation, the measured development of the absorption at the used wavelength corresponds to the volume pulse development of the blood constituent to be determined. To allow for a precise measurement of the volume pulse development, the selection of the used wavelength has to be carried out according to the already described criteria.

An independent invention relates to a method for operating an apparatus for determination of the concentration of different blood constituents. In this connection, use can be made of an apparatus as described in the present application. It is an essential feature that said apparatus comprises at least one radiation source adapted to emit a plurality of measurement radiations of different wavelengths. In the method of the invention, the radiation source will be switched on, preferentially sequentially, for emitting a measurement radiation of respectively one wavelength. This is to say that the radiation source is driven in such a manner that, each time sequentially, it will emit a measurement radiation of a specific wavelength. Particularly, in this connection, the radiation source can be formed of a plurality of individual radiation sources, such as e.g. LEDs, for emitting a measurement radiation of respectively one wavelength. By way of alternative to a sequential emission of a measurement radiation of respectively one wavelength, there can be provided a radiation source which is adapted for simultaneous emission of measurement radiations of different wavelengths. The first and second radiation receiver used herein have to be configured to the effect that they are suited for separate reception of the measurement radiations of the individual emitted wavelengths. This can be realized e.g. by providing a plurality of individual radiation receivers of which each, e.g. with the aid of a frequency filter, receives a specific frequency band of the emitted radiation. It is however preferred that each wavelength is emitted sequentially.

Further, in the method of the invention, the measurement radiation of each wavelength as reflected by a body part to be examined will be received by a first radiation receiver. Further, the measurement radiation of each wavelength transmitted through the body part to be examined will be received by a second radiation receiver. Thereafter, the absorption of the emitted radiation caused by the body part to be examined will be determined for each wavelength. This determination is performed on the basis of the measurement of the reflected radiation by the first radiation receiver and the measurement of the transmitted radiation by the second radiation receiver.

Otherwise, the method of the invention can comprise all of the features which were described in connection with the apparatus of the invention.

Particularly, the method preferably comprises the storing of the detected absorption values for each wavelength and the repetition of the method steps mentioned so far, wherein the detected absorption values of each wavelength will be stored for each repetition cycle. Thereafter, the individual absorption values of the emitted radiation will be combined for each wavelength so as to obtain a representation of a temporal development of the absorption at each wavelength. This representation can be rendered e.g. in the form of a curve or a table and is referred to as the volume pulse development.

The storing of the determined absorption values at each wavelength for each repetition cycle, the combining of the absorption values for each wavelength for representing a temporal development of the absorption, and the representing of said development are preferably performed in a computation device in such a manner that said computation device will not be not in any interdependency with the body part to be examined or with the body of a patient.

To sum up, the method of the invention can comprise the following steps:
a. sequentially radiating a plurality of measurement radiations of respectively different wavelengths,
b. receiving the measurement radiation of each wavelength as reflected by a body part to be examined, by means of a first light receiver,
c. receiving the measurement radiation of each wavelength as transmitted through a body part to be examined, by means of a second light receiver,
d. determining the absorption of the radiation as caused by the body part to be examined, on the basis of the measurement of said reflected radiation by means of the first radiation receiver and the measurement of said transmitted radiation by means of the second radiation receiver,
e. multiple repetition of said method steps a. to d. wherein the respective absorption values for each wavelength of the measurement radiation are stored for each repetition cycle,
f. combining the stored absorption values into a representation of the temporal development of the absorption (volume pulse development) for each used wavelength of the measurement radiation.

Particularly, said method steps d. and f. are performed in a computation device in such a manner that said computation device is not in any interdependency with the body part to be examined.

Preferred embodiments of the invention will be explained hereunder with reference to the Figures.

The Figures show the following:

FIG. 1 a survey of the composition of the human blood,

FIG. 2 a schematic representation of a suitable apparatus for determining the concentration of blood constituents,

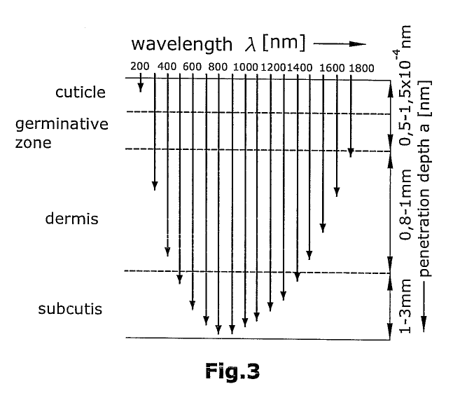

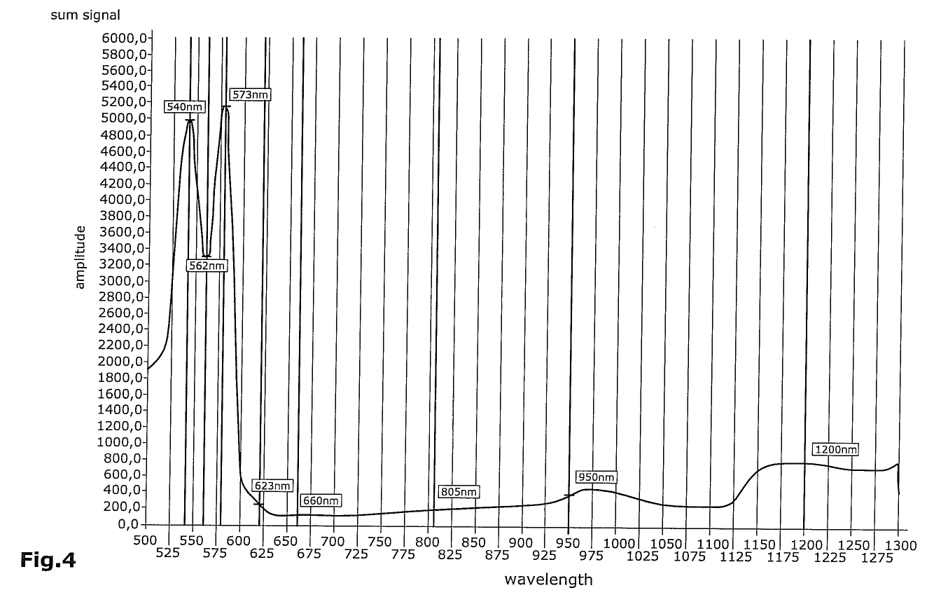

FIG. 3 a graphical representation of the penetration depth of optical radiation into the human skin,

FIG. 4 a graphical representation of the absorption spectrum in human blood at a normal hemoglobin concentration (150 gr/l),

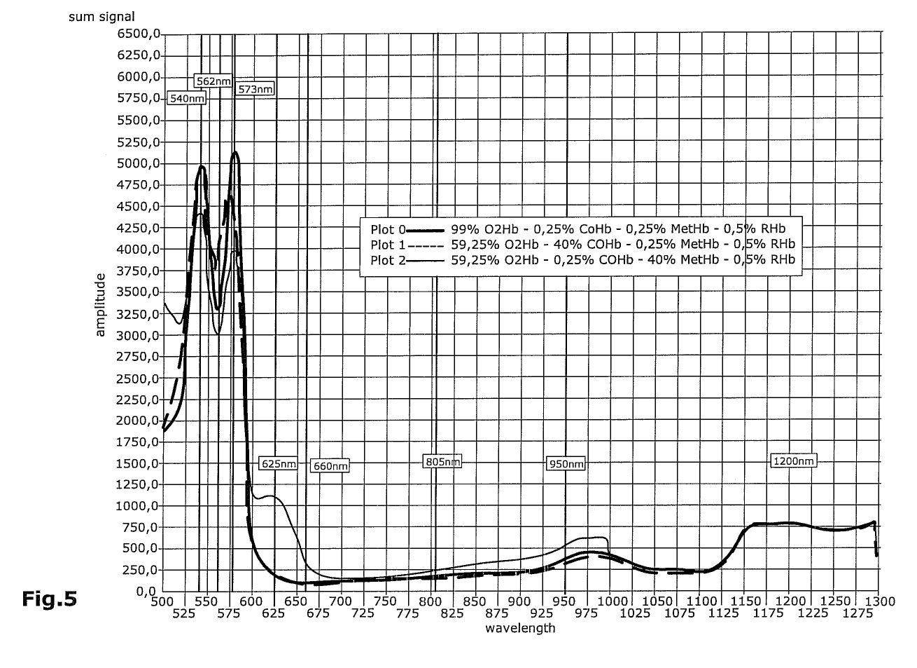

FIG. 5 a graphical representation of a comparison between a normal absorption spectrum and an absorption spectrum at an increased carboxy and respectively hemoglobin concentration,

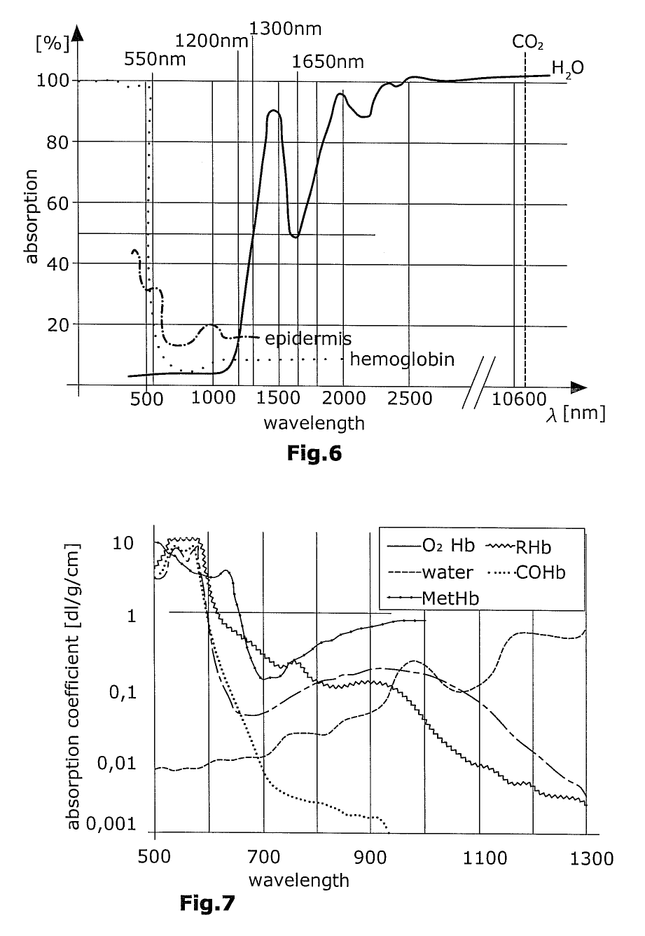

FIG. 6 a graphical representation of the absorption spectrum of hemoglobin and water,

FIG. 7 a graphical representation of the absorption coefficients of various hemoglobin derivates in dependence on the wavelength,

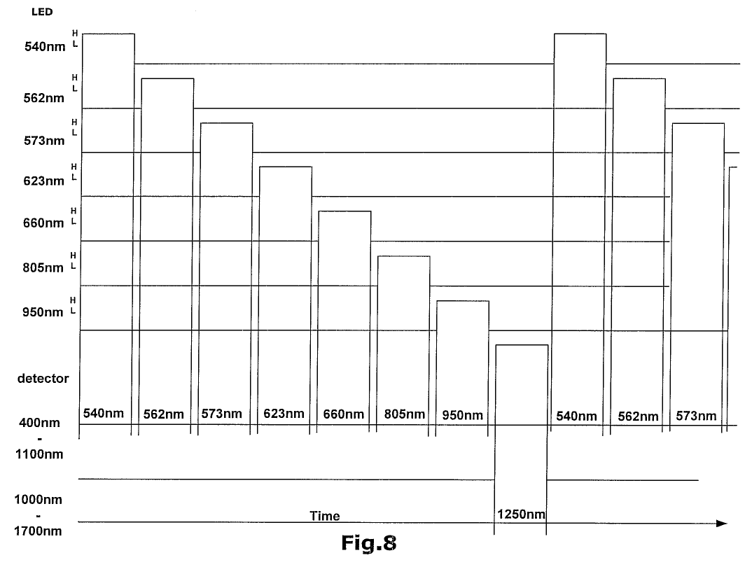

FIG. 8 a graphical representation of the controlling of a plurality of individual radiation sources and of the radiation receivers,

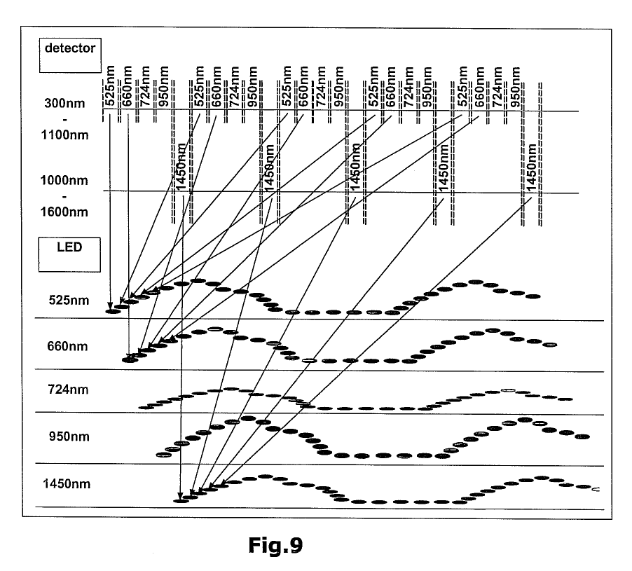

FIG. 9 a schematic representation of the read-out behavior of the radiation receivers,

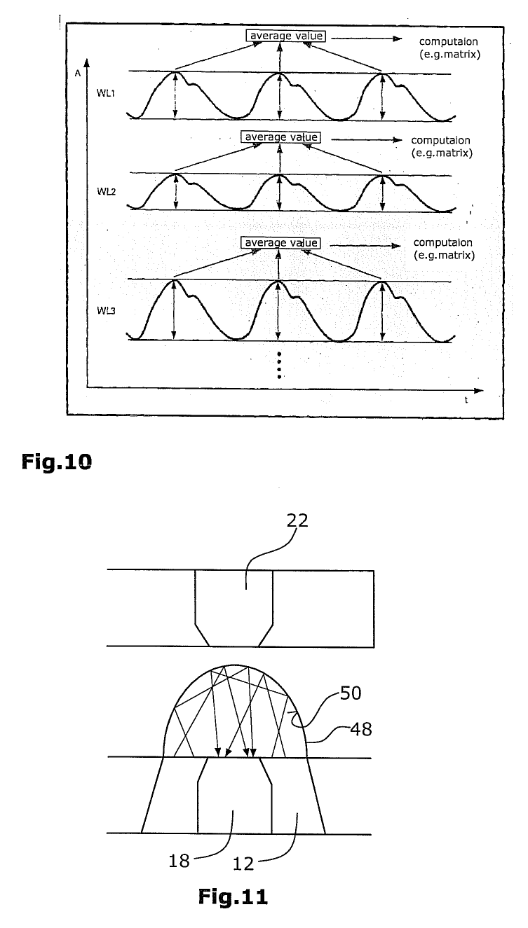

FIG. 10 a graphical representation of absorption developments at different wavelengths,

FIG. 11 a schematic representation of a calibration device for the reflective radiation receiver,

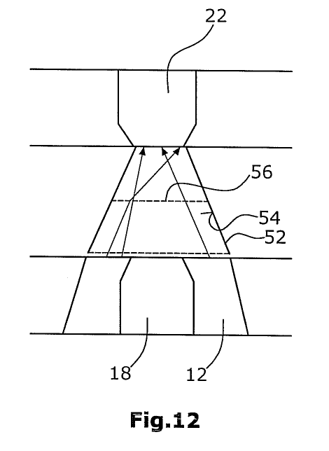

FIG. 12 a schematic representation of a calibration device for the trans-missive radiation receiver,

FIG. 13 a graphical representation of the light intensities for calculating the factors for normalizing the volume pulse curves,

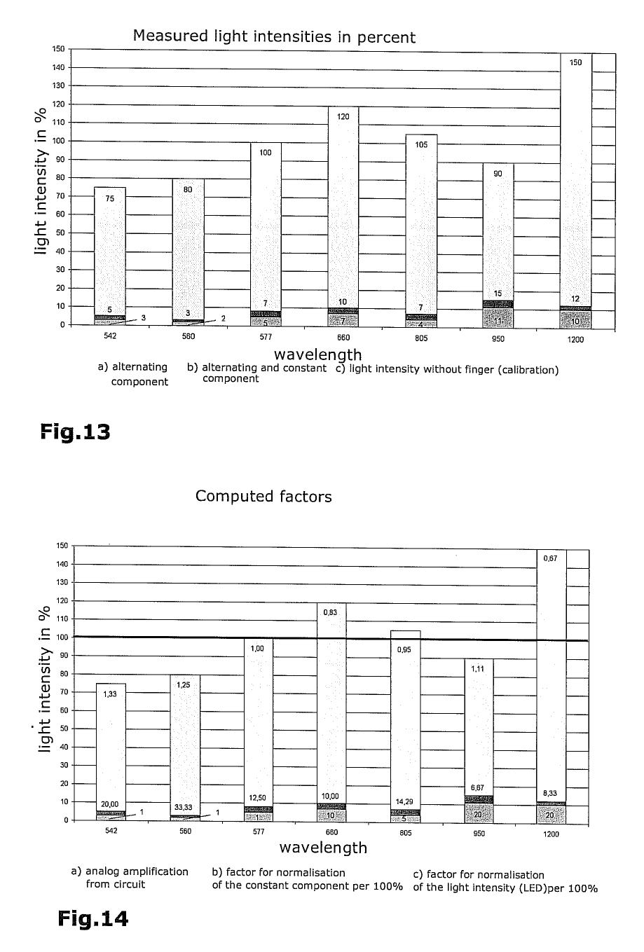

FIG. 14 a graphical representation of the calculated factors for norming the volume pulse curves, and

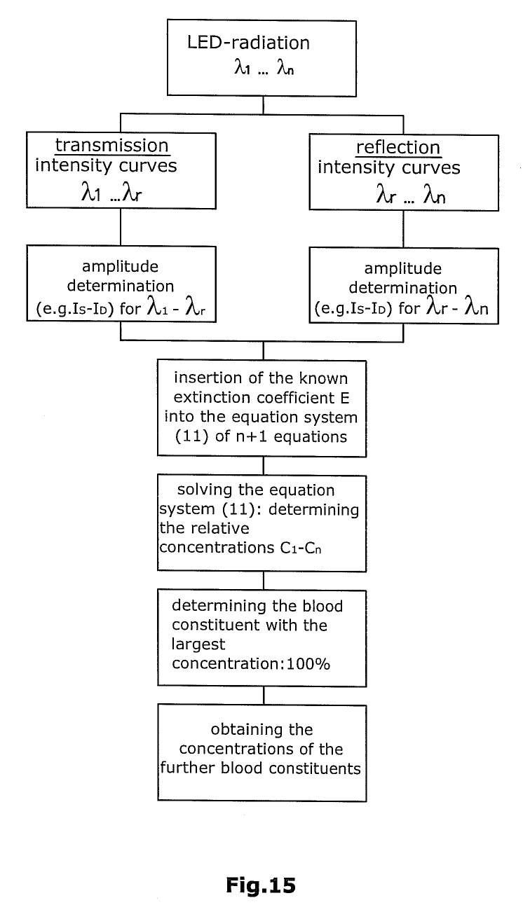

FIG. 15 a process diagram for the determining of the concentration according to a linear equation system,

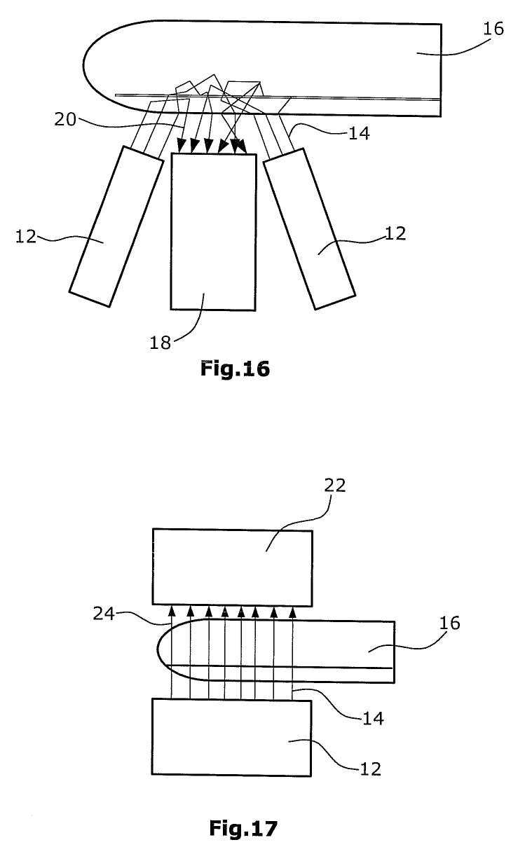

FIG. 16 a schematic representation of a reflection measurement,

FIG. 17 a schematic representation of a transmission measurement,

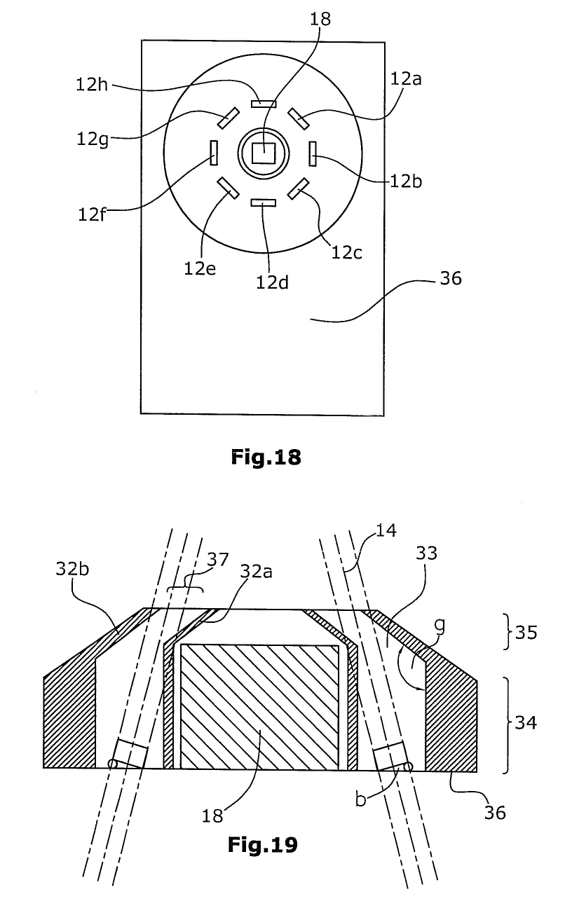

FIG. 18 a schematic representation of the first accommodation element of the apparatus of the invention,

FIG. 19 a sectional view of the first radiation receiver,

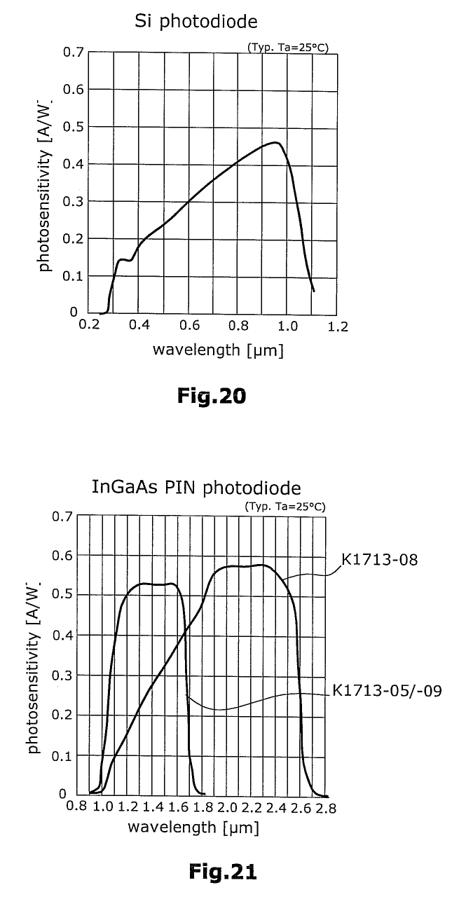

FIGS. 20 and 21 a graphical representation of the detecting ranges of the radiation receivers, and

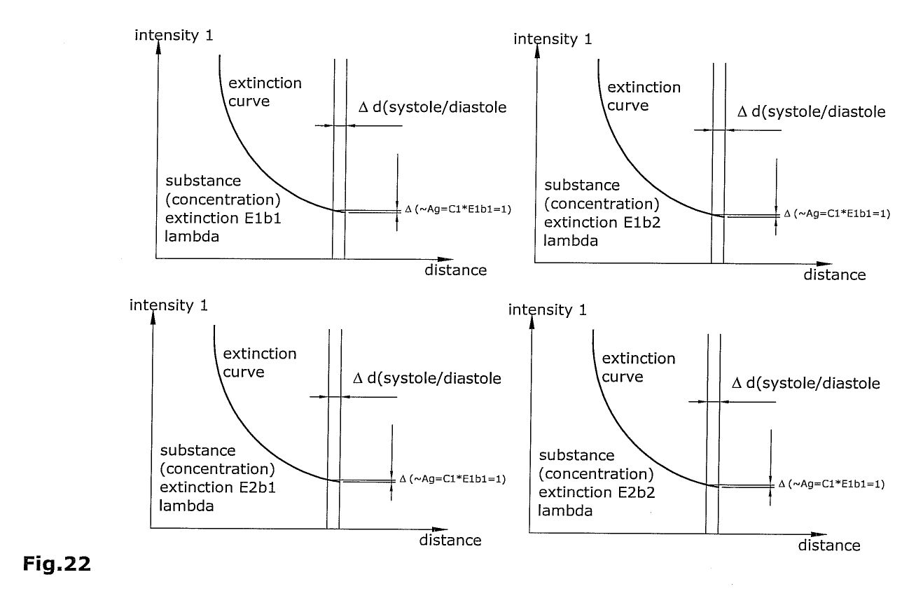

FIG. 22 exemplary extinction curves for two substances at two different wavelengths λ, there being represented the intensity difference ΔI caused by small thickness variations Δd (blood pulsations).

According to FIG. 2, a suitable apparatus for performing the method of the invention comprises a radiation source 12 for emitting a measurement radiation 14 towards a body part 16 to be examined. Preferably, the body part 16 to be examined is a human finger. Alternatively, however, also e.g. the earlobe of a person as well as other suitable body parts can be used for measurement.

Further, said apparatus 10 comprises a first radiation receiver 18 arranged to receive radiation 20 reflected by the body part 16 to be examined. In the illustrated embodiment, the first radiation receiver is arranged in the first accommodation element 28. In said first accommodation element 28, also the radiation source 12 is arranged.

Apparatus 10 further comprises a second radiation receiver 22 arranged to receive radiation 24 transmitted through the body part 16 to be examined. In the illustrated embodiment, the second radiation receiver 22 is arranged in the second accommodation element 30 which is located opposite to first accommodation element 28. “Opposite” in this context means that the two accommodation elements 28,30 as well as the first 18 and the second 22 radiation receiver are arranged in a manner allowing e.g. a finger 16 to be positioned between them.

The emitted measurement radiation 14 will be at least partially reflected by the body part 16 to be examined so that a fraction of the measurement radiation 14 will be reflected as reflected radiation 20 towards the first radiation receiver 18.

At least a fraction of said radiation 14 will pass through the body part 16 to be examined and will be incident as transmitted radiation 24 onto the second radiation receiver 22. The first 18 and the second 22 radiation receiver are preferably designed as photodiodes.

The apparatus further comprises a computation device 26 connected to the first 18 and the second 22 radiation receiver. The measured reflected 20 and the transmitted 24 fraction of the radiation are supplied to said computation device 26 so that the latter, on the basis of the measurement radiation fractions, can determine the absorption of the emitted radiation 14 caused by the body part 16 to be examined.

Computation device 26 can be designed e.g. as a PC operated by a specific software program for performing said computations. Particularly, said computations can be performed on a PC also at a time different from that of the measurement of the transmitted and reflected radiation. Thus, the computational steps which are essential for the invention are performed independently of the physical detection of the patient's features which have been described up to now.

The method of the invention can be controlled particularly by a control device 41, such as e.g. a computer or a microprocessor. Said control device 41 can be a part of apparatus 10.

For performing the method of the invention, also other suitable apparatus can be used. It is of essence herein that such an apparatus comprises at least one radiation source for emitting a measurement radiation with different wavelengths.

The emitted wavelengths are in a range where e.g. the human skin is permeable to radiation. This range is called an optical window and is in a wavelength range of about 350 nm to 1650 nm (see FIG. 7). Outside said range, the absorption of the skin is so high that hardly any radiation can still enter the tissue thereunder.

As illustrated in FIG. 6, it is at these decisive wavelengths that e.g. hemoglobin derivates as well as the water in the human blood will show particularly significant differences with respect to the degree of absorption.

It is particularly preferred, for instance, that the light source 12 comprises a plurality of LEDs adapted to emit different wavelengths. In this case, the LEDs can be controlled in such a manner that the LEDs will be successively switched on and off, e.g. with a frequency of 1.2 kHz, so that no two wavelengths which are different from each other will be emitted simultaneously. A time-parallel emission of measurement radiations of different wavelengths is possible, too, if a plurality of radiation receivers are provided which, e.g. by means of frequency filters, will be able to receive only one specific frequency band of the radiation. What is preferred, however, is a sequential activation of the individual radiation sources, wherein, apart from a frequency of 1.2 kHz, also a range of further suitable frequencies can be used.

The representation of the received signals can be performed either in the so-called normal operation or in the lock-in operation. The lock-in operation will lead to an improvement of the signal quality. In order to allow for the lock-in method to be applied, the lock-in amplifier has to detect the signal one time when the respective LED is in the switched-on state, and one time when the LED is in the switched-off state. The clock-pulse rates of the LEDs can thus be different in normal operation and lock-in operation. In lock-in operation, the control frequency for the LEDs can be adapted to the required frequency of the lock-in amplifier.

The lock-in principle is a method for filtration and amplification of very small signals. In this method, a reference signal of known frequency and phase is modulated onto the measurement signal so that direct voltages of other frequencies as well as noise will be eliminated.

Both in normal operation and in lock-in operation, a constant component (constant portion/offset) as well as a pulse-shaped alternating component (alternating portion) are determined. The constant component is conditioned by the physiological properties of the irradiated tissue. It is influenced by various causes, such as e.g. the properties of the tissue, the blood vessels without pulsatile portion (venules etc.). On this offset, the pulse-shaped alternating component (alternating portion) is situated which results from the change of volume of the blood.

The DC component can be considered as a correction portion. An additional option resides in the use of analog or digital filters for separating the AC and constant components. In principle, this is possible in both modes but should be carried out at least during normal operation so as to achieve a sufficient signal quality since, otherwise, also all disturbances are amplified.

Shown in FIGS. 8 and 9 is the clock behavior of several LEDs and the clock behavior of the photodiode 18 receiving the reflected light as well as the photodiode 22 receiving the transmitted light. In this regard, only five used wavelengths are shown in FIG. 9. In a corresponding manner, the principle of the read-out behavior according to FIG. 9 can also be transferred to a larger or smaller number of wavelengths. Preferred is a use of eight different wavelengths as are shown by way of example in FIG. 8.

Illustrated is a so-called sample-and-hold operation wherein the lower half of the Figure shows two detector signals of the two used detector surfaces for respectively one wavelength range. Herein, a silicon receiver surface will cover the wavelength range from 400 nm to 1100 nm while an indium gallium arsenide receiver surface will cover the wavelength range from 1000 nm to 1700 nm. Upon switch-on of the LEDs with 540, 562, 573, 623, 660, 805 and 950 nm, there will be detected each time the reflected 20 and the transmitted 24 measurement radiation by means of the silicon receiver surface. The value of the signal applied to the detector at each point of time will be picked up for each wavelength (sample), retained (hold) and stored until the wavelength will be activated again. A corresponding process is performed under inclusion of the LED with 1250 nm and of the indium gallium arsenide sensor into the same clock regime. This sequence will be repeated so that the individual samples (values) can be further processed. As shown in FIG. 9, this is performed successively for each wavelength.

Thus, in the first cycle, e.g. eight wavelengths are emitted, their reflected 20 and their transmitted 24 light fraction are measured and then stored. With the aid of the further read-out values from the following repetition cycles, the individual absorption values of the emitted radiation 14 for each wavelength are combined so as to obtain a representation of a temporal development of the absorption at each wavelength.

A first option for determining the concentration ratios resides in the use of linear equation systems.

Starting from equation (1), the following applies approximately (Taylor series) for small deviations (d−d₀) of the layer with the thickness d₀ permeated by the radiation:

I/I₀=10^{-ECd0}−2,3EC(d−d₀)+ (2)
d layer thickness (total)
d₀ layer thickness (total) at a specific point of time (e.g. during the diastole)

In case that the changes of the layer thickness d are caused only by the blood pulsations in the tissue, the value of (d−d₀) is so low that the occurring error will be very small even when using only the linear member of the series development. According to equation (2), the transmitted intensity will result from the constant value 10^{-ECd0} as well as from a usually considerably smaller portion which is caused by the pulsatile change of the diameter of the tissue permeated by radiation.

Preferably, according to the invention, the intensity of the transmitted light wave will be measured during the systole (Iₛ) and the diastole (I_{d}), and the intensity difference will be obtained. It is assumed that the signal of the phototransistor is proportionate to the incident intensity.

On the basis of equation (2), there follows:

(Iₛ−I_{d})/I₀=10^{-ECd0}−2,3EC(dₛ−d₀)−{10^{-ECd0}−2,3EC(d_{d}−d₀)} (3)

(Iₛ−I_{d})=−2,3EC(dₛ−d_{d})I₀ (4)

This means that the determined intensity difference between the systole and the diastole is proportionate to the molar extinction E and the concentration C of the blood constituent and to the path difference (dₛ−d_{d}).

The factor EC is a measure for the absorption A of the transmitted light.

A−EC (5)

The absorption at a specific light wavelength is directly proportionate to the substance concentration of the observed blood constituent and is proportionate to the absorption coefficient of the blood constituent at the given wavelength, i.e. the higher the substance concentration per given volume or the higher the value of the absorption coefficient is, the stronger the absorption will be.

For specific wavelengths A and concentrations C_{bn} of the blood constituents, the following applies:

A_{bn}=E(λ,bn)*C_{bn} (6)

On the basis of equation (4), the interrelation between the absorption A and the measured intensity difference ΔI=(Iₛ−I_{d}) can be obtained. As of yet, it has been assumed that the entire irradiated intensity minus the absorption in the tissue will arrive at the receiver. This is not the case in practice. Moreover, also the thickness difference (dₛ−d_{d}) is always unknown. For this reason, the absorption can be accurately determined only except for an unknown factor K_{T} (the constant for transmission measurement).

ΔI=K_{T}A (7)
ΔI intensity difference between systole and diastole with respect to the light exiting from the body part
K_{T} constant for the interrelation between the measured intensity and the absorption at a transmission measurement It is of importance that this factor is (practically) constant for all measurements at all wavelengths.

The example of a computation has up to now been rendered for measurements in transmission. In practice, it becomes evident that, at specific wavelengths, the absorption is very high and the signal/noise ratio is unfavorable. In such cases, measurement in reflection is better.

In measurement in reflection, this factor will differ from that of the transmission measurements. For reflection measurements, the following applies:

ΔI=K_{R}A (8)
K_{R} constant for the interrelation between the measured intensity and the absorption at a reflection measurement It is assumed that the absorption will add up for the individual blood constituents b1 . . . bn. This regularity does not exist in case of too high concentrations C.

Thus, at a given wavelength λ, the following applies for the total absorption Ag:

Ag=A_{b1}+A_{b2}+ . . . +A_{bn} (9)
A_{g} total absorption
A_{b1} absorption of blood constituent 1
A_{b2} absorption of blood constituent 2
A_{bn} absorption of blood constituent n

In connection with equation (6), what follows is:

Ag(λ)=E(λ,b1)*C_{b1}+E(λ,b2)*C_{b2}+ . . . +E(λ,bn)*C_{bn} (10)
E(λ,bn) absorption coefficient of blood constituent n at light wavelength λ
C_{bn} concentration of blood constituent n

Known in each case are the measured total absorption Ag (except for a factor K_{T} and resp. K_{R}) for each of the used light wavelength, and the respective absorption coefficient belonging to the blood constituent at the light wavelength used. The respective portions of the substance concentrations are unknown and have to be computed.

Now, corresponding to equation (10), an n*n equation system can be established for determining the individual portions of the blood constituents relative to the total volume.

For instance, in a case where 5 different concentrations have to be determined, measurements must be performed with 5 different light wavelengths in order to be able to solve the equation. This will result in a linear 5×5 equation system which can be precisely solved. However, if the measurements are to be performed partially in transmission and partially in reflection, there will be required a further equation for determining a factor (K_{T}/K_{R}) which represents the different conditions in the measurements in transmission and reflection.

If, for instance, the measurements at the wavelengths λ₁ to λ₃ have to be performed in transmission, and the measurements at the wavelengths λ₃ to λ₅ in reflection, this will result in the following system of equations (11). Therein, the measurement at the wavelengths λ₃ is performed both in transmission and in reflection:

Ag_{T}(λ1)=E(λ₁,b₁)*C_{b1}+E(λ₁,b₂)*C_{b2}+E(λ₁,b₃)*C_{b3}+E(λ₁,b₄)*C_{b4}+E(λ₁,b₅)*C_{b5}

Ag_{T}(λ2)=E(λ₂,b₁)*C_{b1}+E(λ₂,b₂)*C_{b2}+E(λ₂,b₃)*C_{b3}+E(λ₂,b₄)*C_{b4}+E(λ₂,b₅)*C_{b5}

Ag_{T}(λ3)=E(λ₃,b₁)*C_{b1}+E(λ₃,b₂)*C_{b2}+E(λ₃,b₃)*C_{b3}+E(λ₃,b₄)*C_{b4}+E(λ₃,b₅)*C_{b5}

Ag_{R}(λ3)=(K_{T}/K_{R})*{E(λ₃,b₁)*C_{b1}+E(λ₃,b₂)*C_{b2}+E(λ₃,b₃)*C_{b3}+E(λ₃,b₄)*C_{b4}+E(λ₃,b₅)*C_{b5}}

Ag_{R}(λ4)=(K_{T}/K_{R})*{E(λ₄,b₁)*C_{b1}+E(λ₄,b₂)*C_{b2}+E(λ₄,b₃)*C_{b3}+E(λ₄,b₄)*C_{b4}+E(λ₄,b₅)*C_{b5}}

Ag_{R}(λ5)=(K_{T}/K_{R})*{E(λ₅,b₁)*C_{b1}+E(λ₅,b₂)*C_{b2}+E(λ₅,b₃)*C_{b3}+E(λ₅,b₄)*C_{b4}+E(λ₅,b₅)*C_{b5}}
Ag_{T}(λn) total absorption for transmission at the wavelength λn
Ag_{R}(λn) total absorption for reflection at the wavelength λn

Equation system (11) comprises the 5 concentrations C_{bn} as well as the factor (K_{T}/K_{R}) as an unknown value and thus can be clearly solved. If measurements are to be performed exclusively in transmission or reflection, the number of equations is reduced to the number n of the wavelengths. On the other hand, also a redundancy in determination, with more equations than unknown values, is possible for increasing the precision of the result. By solving the equation system (11) via matrices or substitution and inserting the respective coefficients and the results of the absorption measurement, one will directly obtain the discrete substance concentrations C_{b1} to C_{b5}.

It will be understood that the equation system (11) is based on the precondition that the intensity I₀ irradiated into the body part as well as the sensitivities of the photodetector are identical for all wavelengths or that a corresponding normalization has been performed. In this regard, reference is made to FIG. 14 and the respective explanations.

Since the measurements of the absorptions Ag(λ) on the basis of the intensity differences ΔI can be performed accurately only with exception of the unknown constants K_{T} and K_{R}, there is missing a parameter for determining the absolute value of the concentration. Thus, it is agreed that the measure 100% will be assigned to the highest concentration of substance concentration. However, by determining the main blood constituents inclusive of the water fraction, the composition of the blood will become known except for an inaccuracy of a few percent.

An exemplary process diagram for computing the concentrations of the blood constituents is shown in FIG. 15.

It should be mentioned that, in case of measurements in reflection operation, it will normally be required to perform corrections because the conditions are more complex than in transmission operation. Particularly, consideration has to be given to the different scattering of the light for different wavelengths at the blood constituents.

A second possibility for determining the concentration ratios is offered by the heuristic great deluge algorithm.

Use is made of eight different wavelengths wherein, each time, the appertaining total absorption will be measured. What is important herein is not the absolute measurement values but the mutual relations of the measurement values used in the great deluge algorithm. The measurement values are scaled in such a manner that the highest absorption will correspond to 100%. Consequently, each further absorption of the remaining 7 wavelengths will have a value smaller than 100%.

Now, there shall be assumed a first random blood composition which is theoretically possible.

Based on the knowledge of the substance-specific and wavelength-specific absorption coefficients of the involved blood constituents, there is computed a theoretically expected total absorption for each wavelength. Exactly as in real measurement, also the theoretical absorptions will be scaled to 100%. This first computed absorption spectrum will be correlated with the real measured absorption spectrum. The correlation coefficient resulting therefrom is the starting point of the algorithm.

Now, at each round, the previous theoretically assumed blood composition will be slightly changed, and the resultant absorption spectrum will again be correlated with the measured absorption spectrum. This new correlation coefficient will be compared to a threshold value which slightly increases per round.

If the threshold value exceeds the current correlation coefficient, the newly determined theoretical blood composition is discarded, and, on the basis of the previous one, a new blood composition is determined.

If the threshold value does not exceed the current correlation coefficient, the threshold value is slightly increased, and the newly determined blood composition will be used as a starting point of the next round.

If no change of the blood composition is possible anymore that would not lead to a transgression of the current threshold value, the algorithm is stopped.

It can be assumed that the current blood composition corresponds to a good approximation to the real measured blood composition.

The algorithm can be carried out a plurality of times with different marginal parameters and different starting points for better verification of the heuristic result.

According to the deluge method and by use of slightly varied parameters, a search is performed for neighboring solutions which, in case of a worse adaptation to the measurement value, will still be accepted exactly when they exceed a threshold value referred to as a “water level”. In the course of the method, this threshold value is—starting from zero—continuously increased until no improvement of the current solution can be achieved anymore. This method leads to good approximations with relatively little computational expenditure.

The concentration ratios can further be determined by correlation.

Based on the knowledge of the substance- and wavelength-specific absorption coefficients and under the assumption of a theoretically possible blood composition, it is possible to compute a corresponding total absorption which is to be expected at the individual light wavelengths. In this regard, the relationship of the absorptions relative to each other is decisive again.

There is computed a set of such theoretically conceivable absorption spectra wherein each to-be-determined substance concentration is represented in small steps respectively from the minimal possible portion to the maximal possible portion. In our case, these are:
water 44-54%
oxygenated hemoglobin 50-100%
non-oxygenated hemoglobin 1-50%
carboxy hemoglobin 1-60%
methemoglobin 1-70%

The measured absorption spectrum will now be correlated with all spectra that have been calculated in advance in this manner. The theoretically determined concentration ratios of the blood constituents of the spectrum which best correlates with the measured spectrum, correspond to the real concentration ratios with good approximation.

The precision of this method primarily depends on the available computing power of the hardware. The more computing power is available, the more finely the concentration grades can be selected, and the more accurate the expected result will be.

The advantage of this method over the great deluge algorithm is that the result will be unambiguous and not of a heuristic nature, which means that, under consideration of a certain imprecision of the result that is unavoidable due to a step width larger than zero in the distribution of parameters, there will surely be no possibly better result than the one which has been determined.

In the great deluge algorithm, it cannot be assumed with certainty that the detected result corresponds to the best result. To compensate for this, the algorithm will be practiced a plurality of times with different starting points.

The advantage of this method resides in that, by the reducing of the step width of the parameters briefly before the end of the algorithm, the determined result can represent a relatively more-accurate result than the method of the correlation.

The starting point of the explained computation methods resides in the volume pulse curves which have been detected for each wavelength (FIG. 10). The curves will be subdivided into individual heart periods, and for each period, the maximum will be determined at each wavelength. For each wavelength, there will then be calculated an average value of the maxima (amplitudes). Thus, depending on the number of the used wavelengths, five or six average absorption values are obtained which have to be considered as relationships to each other. With the aid of the computation methods mentioned above, the pro-rata concentration of the blood constituents can be computed. From these concentrations, one can determine further parameters, such as e.g. oxygen saturation, total hemoglobin concentration or the hematocrit value.

To achieve a general improvement of the measurement results, it is possible to calibrate the apparatus at different time intervals. For this purpose, use can be made of a first calibration device 48 according to FIG. 11 which is configured e.g. as a calibration shell for the reflective receiver. For determining the real light intensities of the LEDs, the calibration of the apparatus is first performed without the body part to be examined.

For calibrating the reflective radiation receiver 18, a semispherical calibration shell 48 can be positioned on the reflective sensor 18, so that also the emitting surface is included. Said calibration shell 48 comprises a white inner surface 50 which is stimulated by a diffuse reflection of the light so that the light intensity of the individual LEDs can be detected.

Further, according to FIG. 12, calibration of the sensor for the transmitted light fraction 22 use can be made of a preferably frustoconical calibration shell 52 between the receiver for the transmitted light fraction 22 and the reflective receiver 18. Said calibration shell again comprises a white inner surface 54 and further a white membrane 56 placed centrally between the two detector surfaces. Said white membrane 56 prevents a direct radiation from the LEDs onto the receiver for the transmitted light fraction and at the same time generates a non-directional diffuse light radiation. With the aid of this measurement, the light intensities of the respective LEDs are determined in relation to the receiver 22 working in transmission.

FIG. 13c shows the total measured light intensity of each LED. This measurement is referred to as a zero measurement and can be carried out by the above mentioned calibration shells. To make it possible to assume a uniform radiation for further computations, the normalizing of the intensities will be performed to 100%. In the process, a normalization factor for the light intensity is obtained for each LED (FIG. 14c). This calibration measurement is performed once for each sensor head and is carried out at defined time intervals, e.g. each two or three years. This method is necessitated by the decrease of the decreasing light power of the light sources used, e.g. of the LEDs.

After the above described calibration, the measurement can be performed on a body part. The now following method steps can belong to the method of the invention individually or in their entirety. Preferably, a DC component and an AC component will be detected. To make it possible to compare the detected pulsatile light absorptions, they have to be normalized in dependence on the DC component for each wavelength. For this purpose, a determination of the DC component for each wavelength is performed at least once per measurement.

By way of example, FIG. 14b shows the transmitted light fraction. As represented in the Figure, the by far largest fraction of the light has been absorbed by the tissue (bones, skin and its constituents, e.g. melanin, degradation products of hemoglobin, z. B. bilirubin, venules etc.).

For the light intensities measured during the calibration without finger (FIG. 13c), light intensity factors (1.33; 1.25; 1.00 etc.) will be detected, as shown in FIG. 14. Further detected are factors for normalizing the DC components to 100% (in FIG. 14: 20.00; 33.33; 12.5; etc.).

The computation is performed as represented in the following table:

| | wavelength | | | | | | |
|---|---|---|---|---|---|---|---|
| | 542 | 560 | 577 | 660 | 805 | 950 | 1200 |
| | nm | nm | nm | nm | nm | nm | nm |
| measurement | 3 | 2 | 5 | 7 | 4 | 11 | 10 |
| of the AC frac- | | | | | | | |
| tion by peak | | | | | | | |
| detections from | | | | | | | |
| the pulse curve | | | | | | | |
| in percent | | | | | | | |
| multiplication | 4.00 | 2.50 | 5.00 | 5.83 | 3.81 | 12.22 | 6.67 |
| by light intensi- | | | | | | | |
| ty factor | | | | | | | |
| multiplication | 80.00 | 83.33 | 62.50 | 58.33 | 54.42 | 81.48 | 55.56 |
| with DC frac- | | | | | | | |
| tion factor | | | | | | | |
| division by | 80.00 | 83.33 | 62.50 | 5.83 | 10.88 | 4.07 | 2.78 |
| analog amplifi- | | | | | | | |
| cation | | | | | | | |
| absolute intensi- | 80.00 | 83.33 | 62.50 | 5.83 | 10.88 | 4.07 | 2.78 |
| ty of pulsatile | | | | | | | |
| alternating | | | | | | | |
| component | | | | | | | |

At a wavelength of 542 nm, for instance, a peak value 3 AU is measured (random unit). This peak value will be multiplied by the light intensity factor (herein: 1.33) so that the result 4 AU will be obtained. Thereafter, this result will be multiplied by the constant component factor (herein: 20). The obtained 80 AU will then be divided by the analog amplification of 1, wherein each wavelength is characterized by its own amplification (according to FIG. 14: 542 nm: 1; 560 nm: 1; 577 nm: 1; 660 nm: 10; 805 nm: 5; 950 nm: 20; 1200 nm: 20).

The absolute intensity of the pulsatile AC component at 542 nm will thus be 80.00 AU. For the rest of the wavelengths, the computation of the AC components will be performed in accordance with the same principle so that the normalized values can be compared to each other. By this methodic approach, the DC components are removed, and only the portions undergoing pulsatile variation will be considered. The DC component has an individual value for each human, due his/her skin color, the condition of his/her skin (cornification), his/her skeletal structure and other properties which depend on the measurement site.

However, as explained above, also other computational methods can be applied in the method of the invention.

Further details of various embodiments of a suitable apparatus for determination of concentrations of different blood constituents will be described hereunder with reference to FIGS. 16 to 21.

FIG. 16 shows a schematic representation of the reflection measurement performed with the aid of an embodiment of the apparatus of the invention. In the process, the radiation source 12 emits a measurement radiation 14. Said radiation source 12 can be formed, as preferred, as a plurality of LEDs 12a to 12h. The emitted measurement radiation 14 is at least partially reflected by the body part 16 to be examined, so that a fraction of the measurement radiation 14 will be reflected as reflected radiation 20 towards the first radiation receiver 18.

The measurement of the radiation 24 transmitted by the body part 16 to be examined is schematically represented in FIG. 17. Also in this case, the radiation source 12 emits a measurement radiation 14 towards the body part 16 to be examined. At least a fraction of the radiation 14 will pass through the body part 16 to be examined and will be incident as transmitted radiation 24 on the second radiation receiver 22. The first 18 and the second 22 radiation receiver are preferably designed as photodiodes.

Further, as shown in FIG. 2, the apparatus comprises a computation device 26 connected to the first 18 and the second 22 radiation receiver. The measured reflected 20 and transmitted 24 fraction of the radiation are supplied to said computation device 26 so that this device, on the basis of the measurement radiation fractions, can determine the absorption of the emitted radiation 14 caused by the to-be-examined body part 16.

Computation device 26 can be designed e.g. as a PC with a specific software program running thereon for performing the above mentioned computations. Particularly, on a PC, these computations can be carried out at a different time than the measurement of the transmitted and reflected radiation. Thus, the computation steps which are essential of the invention are performed independently from the physical detection of the patient features described up to now.

With particular preference, the apparatus 10 according to FIG. 18 is designed to the effect that the radiation source 12 comprises a plurality of individual radiation sources 12a to 12h. These individual radiation sources can be formed as LEDs and be arranged in a circular configuration around the first radiation receiver 18.

As shown in FIGS. 18 and 19, the first light receiver 18 is arranged within a preferably circular separating means 32 which can comprise an inner light-impermeable shell 32a as well as an outer light-impermeable shell 32b having an inner wall with a white coating. In this configuration, the LEDs 12a to 12h are arranged in an intermediate space 33 between the inner shell 32a and the outer shell 32b. On their side facing away from the first light receiver 18, the LEDs are lifted by an angle of 15°. Thereby, the emitted measurement radiation will be bundled at a point where the body part 16 to be examined is arranged.

Preferably, in a lower portion 34, the inner 32a and the outer shell 32b extend—starting from a base plate 36 such as e.g. a circuit board—vertically upwards and then are bent inwards, in an upper portion 35, at an angle β, i.e. towards the first radiation receiver 18. By this arrangement in combination with the lifted orientation of the LEDs, e.g. at angle of 15 degrees, it is safeguarded that only a narrow gap 37 is available through which the emitted measurement radiation 14 can radiate towards the body part 16 to be examined. Thereby, it can be effectively avoided that stray light (shunt light) will radiate directly from the light source 12 towards the first radiation receiver 18. The objective of this measure is that the first radiation receiver 18 will receive only the radiation 20 which is reflected by the body part 16 to be examined.

Between the inner shell 32a and the outer shell 32b, a cavity 33 is formed within which the LEDs 12a to 12h are arranged e.g. on said circuit board 36. Said cavity 33 can be filled with a transparent adhesive, for instance.

Based on the fact that different hemoglobin derivates as well as the water in the blood will absorb different wavelengths to different extents, the individual light sources 12a to 12h can be configured to emit the following wavelengths:
540 nm±5 nm, 562 nm±5 nm, 573±5 nm
623±5 nm
660 nm±10 nm
805 nm±10 nm
950 nm±10 nm
1200 nm±50 nm

A graphic representation of the detectable wavelength ranges of the receiver surfaces used is found in FIGS. 20 and 21. The characteristic lines shown in FIG. 21 represent two different indium gallium detectors. With particular preference, use is made of the detector on the left (L 1713-05/-09).

Seven of the eight wavelengths will be detected by the silicon detector. Wavelengths above 1100 nm will be detected correspondingly by the indium gallium arsenide photodiode.

According to FIG. 2, it is preferred that the body part 16 to be examined will be accommodated in an accommodation chamber 38 arranged between the first accommodation element 28 and the second accommodation element 30.

The apparatus can further comprise a clamping mechanics 40, e.g. a spring mechanics, which is interconnected by the first 28 and the second 30 accommodation element in such a manner that the device 10 can be applied to the body part 16 to be examined. For easier placement of apparatus 10, e.g. on a finger 16, two actuation projections 42 can be provided.

With particular preference, the apparatus 10 comprises a control device 41 for sequential switch-on of the individual light sources 12a to 12h.

FIG. 22 schematically represents, by way of a simple example with the aid of an equation system, the process of computing the concentration of blood constituents. Preferably, the following method steps will be performed:
1. emitting light of different wavelengths A into the body tissue
2. determining the intensity curves I of the transmitted or reflected light fraction
3. determining the intensity difference Iₛ−I_{d} between systole and diastole
4. inserting into the equation system (11) the known extinction coefficients E for the blood constituents expected in the body tissue
5. solving the equation system (11)
6. setting the blood constituent with the highest concentration to 100%
7. determining the concentration of the further blood constituents

Hereunder, the required computations will be explained by way of a simple example:

From the Lambert-Beer law

I/I₀=10^{-ECd}
I transmitted intensity
I₀ incident intensity
E absorption coefficient (molar extinction) of a blood constituent (for a specific wavelength)
C concentration of the blood constituent
d layer thickness
   there follows, by way of approximation, for small changes Δd of the layer of the thickness d, which is permeated by radiation:

(Iₛ−I_{d})/I₀=−2,3EC(dₛ−d_{d})
dₛ layer thickness (in total) during the systole
d_{d} layer thickness (in total) during the diastole

The product from the molar extinction E and the concentration C of the blood constituent is defined as the total absorption Ag:

Ag=E*C

By comparison of the two above equations, it becomes evident that the total absorption is proportionate to the measurable intensity difference ΔI=(Iₛ−I_{d}) if the irradiated intensity I₀ as well as the path difference Δd=dₛ−d_{d} is identical for all measurements:

ΔI˜Ag

FIG. 22 shows the extinction curve for two different substances at two different wavelengths λ. From the diagrams, it is evident that the intensity change ΔI in case of a very small change of distance Δd is each time so small that the above derived linear approximation becomes plausible.

For the two substances 1 and 2 (blood constituents b₁ and b₂), the extinction coefficients for the two wavelengths λ₁ and λ₂ are known:

E(λ₁,b₁), E(λ₂,b₁), E(λ₁,b₂) and E(λ₂,b₂)

It is further known that, in case of not too high concentrations C of the blood constituents, the absorptions for the individual components can be added. From this, there follows

Ag(λ)=E(λ,b₁)*C₁+E(λ,b₂)*C₂
A_{g}(λ)=total absorption (for a specific wavelength)
E(λ,bₙ)=absorption coefficient of the blood component n at the light wavelength λ (known)
Cₙ=concentration of the blood constituent n

For two different wavelengths λ₁ and λ₂, there is obtained the equation system

Ag(λ₁)=E(λ₁,b₁)*C₁+E(λ₁,b₂)*C₂

Ag(λ₂)=E(λ₂,b₁)*C₁+E(λ₂,b₂)*C₂

Using the assumed values (by way of example)

E(λ₁,b₁)=1

E(λ₂,b₁)=2

E(λ₁,b₂)=0.25

E(λ₂,b₂)=1.5

C₁=1

C₂=2

there follows:

Ag(λ₁)=1*1+0.25*2=1.5

Ag(λ₂)=2*1+1.5*2=3

Actually, however, only the molar extinction E and the total absorption Ag are known (for different wavelengths). The concentrations C (which in the constructed example were assumed to be known), however, have to be calculated. This calculation is carried out hereunder.

Under inclusion of the unknown concentrations C₁ and C₂, the equation system will read as follows:

Ag(λ₁)=1*C₁+0.25*C₂=1.5K₀

Ag(λ₂)=2*C₁+1.5*C₂=5K₀
K₀ constant, determined by the irradiated intensity I₀ as well as by the path difference Δd (blood pulsations)

From the first equation, there follows

C₁=1.5K₀−0.25*C₂.

From the second equation, what follows is

2*(1.5K₀−0.25C₂)+1.5C₂=5K₀

C₂=2K₀

By insertion of C₂ into the first equation of the equation system, there is obtained:

1C₁+0.25*2K₀=1.5K₀

C₁=1K₀

As agreed upon, the blood constituent with the highest concentration, i.e. C₂, is set to 100%.

This will result in

C₂=100%

C₁=50%

Thus, the concentration of the blood constituents has been calculated by way of a very simple and easily surveyed example.

1. A method for the non-invasive determination of the concentration of blood constituents, comprising:
radiating a plurality of measurement radiations with respectively different wavelengths by means of a radiation source;
receiving the measurement radiation of a plurality of said wavelengths, as reflected by the body part to be examined, by means of a first light receiver;
receiving the measurement radiation of a plurality of said wavelengths, as transmitted through the body part to be examined, by means of a second light receiver; and
determining the absorption of the measurement radiation of each wavelength, as caused by the body part to be examined, on the basis of the measurement of said reflected radiation by means of the first radiation receiver and the measurement of said transmitted radiation by means of the second radiation receiver
wherein that an absorption Ag_{R} is determined by the first radiation receiver on the basis of the measurement of the reflected radiation of a first portion r to rᵢ of the wavelengths, and an absorption Ag_{T} is determined by the second radiation receiver on the basis of the measurement of the transmitted radiation of a second portion 1 to r of the wavelengths, wherein, for at least one wavelength r, the reflected as well as the transmitted radiation fraction is measured, comprising the further step of:
determining the n concentrations C_{b1} to C_{bn} of blood constituents on the basis of said determined absorptions Ag_{R} and respectively Ag_{T} of the measurement radiation at each wavelength 1 to n, the computation of the concentrations being performed according to the following equation system:
A_{T}(λ₁)=E(λ₁,b₁)*C_{b1}+ . . . +E(λ₁,bₙ)*C_{bn}
Ag_{T}(λ_{T})=E(λᵣ,b₁)*C_{b1}+ . . . +E(λᵣ,bₙ)*C_{bn}
Ag_{R}(λᵣ)=(K_{T}/K_{R})*{E(λᵣ,b₁)*C_{b1}+ . . . E(λᵣ,bₙ)*C_{bn}}
Ag_{R}(λₙ)=(K_{T}/K_{R})*{E(λₙ,b₁)*C_{b1}+ . . . +E(λₙ,bₙ)*C_{bn}}

2. The method according to claim 1, wherein said method steps are repeated a plurality of times and the respective absorption values for each wavelength of the measurement radiation are stored for each repetition cycle.

3. The method according to claim 2, wherein the stored absorption values for each wavelength are combined into a representation of the temporal development of the absorption for each wavelength of the measurement radiation.

4-7. (canceled)

8. The method according to claim 1, wherein the preceding step:
determining a normalization factor for the light intensity of the radiation source.

9. The method according to claim 1, wherein, at least once per measurement, the constant component of the obtained development of the absorption is determined for each wavelength.

10. The method according to claim 9, wherein the determined alternating component of the development of the absorption for each wavelength is normalized in dependence on the determined constant component for each wavelength.

11. The method according to claim 1, wherein analog and/or digital filters are used for separating the DC and alternating components of the detected developments of the absorption at each wavelength.

12. The method according to claim 1, wherein the detected constant component of the developments of the absorption is included as a correction portion.

13. The method according to claim 1, wherein the said radiating of a plurality of measurement radiations with respectively different wavelengths by means of a radiation source is performed sequentially.

14-16. (canceled)
